(19) United States
(12) Patent Application Publication
(10) Pub. No.: US 2016/0032386 A1
(43) Pub. Date: Feb. 4, 2016

(54) GENETIC MARKERS FOR OSTEOARTHRITIS

(71) Applicants: PROGENIKA BIOPHARMA S.A., Derio (ES); BIOIBERICA, S.A., Barcelona (ES)

(72) Inventors: Josep Escaich, Barcelona (ES); Josep Vergés, Barcelona (ES); Ruth Alonso, Barcelona (ES); Eulàlia Montell, Barcelona (ES); Helena Martínez, Barcelona (ES); Marta Herrero, Barcelona (ES); Francisco Blanco, Barcelona (ES); Antonio Martínez, Vizcaya (ES); Diego Tejedor, Derio (ES); Marta Artieda, Derio (ES); Nerea Bartolomé, Derio (ES)

(73) Assignees: BIOIBERICA, S.A., Barcelona (ES); PROGENIKA BIOPHARMA S.A., Derio (ES)

(21) Appl. No.: 14/776,277

(22) PCT Filed: Jan. 30, 2014
(86) PCT No.: PCT/EP2014/051766

§ 371 Date: Sep. 14, 2015

(30) Foreign Application Priority Data
Mar. 14, 2013 (GB) 1304614.9

(51) Int. Cl. C12Q 1/68

(52) U.S. Cl. CPC C12Q 1/6883; C12Q 2600/158; C12Q 2600/156; C12Q 2600/16; C12Q 2600/118

(57) ABSTRACT

A method for predicting the severity or progression of OA in a human subject, comprising: determining the identity of at least one allele at each of at least 4 positions of single nucleotide polymorphism (SNPs) selected from the group consisting of: rs2206593, rs10465850, rs780094, rs1374281, rs1143634, rs2073508, rs2243250, rs4720262, rs917760, rs7838918, rs12009, rs730720, rs874692, rs893953, rs1799750, rs10845493, rs11054704, rs7986347, rs1802536, rs10519263, rs7342880, rs16947882 and rs10413815, and one or more SNPs in linkage disequilibrium at a level of at least R²≧0.8 therewith, as well as products, in particular systems and kits for use in such a method.

### FIELD OF THE INVENTION

The present invention relates to methods for predicting the progression of osteoarthritis, products for use in the methods and related systems.

### BACKGROUND TO THE INVENTION

Osteoarthritis (OA) is a degenerative joint disease, more common among women, which involves deterioration of the cartilage and the subchondral bone, and synovial inflammation. It commonly occurs in the weight bearing joints of the hips, knees, and spine. It also affects the fingers, thumb, neck, and large toe. Knee OA is the most common type of OA and also one of the most common causes of disability. Current therapeutic approaches are insufficient to prevent initiation and progression of the disease.

It is well-accepted that the etiology of OA is multifactorial and that involves genetic and environmental factors (Fernández-Moreno et al. 2008). The genetics of this disease is complex, it does not follow the typical pattern of mendelian inheritance, it is a disease associated with multiple gene interactions. Several studies support the theory of a polygenic inheritance, as opposed to defect in a single gene (Panoutsopoulou et al. 2011, Meulenbelt 2011). Epidemiological studies estimate that the influence of genetic factors in radiographic OA of the hip or knee in women is 60% and 39%, respectively, independent of known environmental or demographic confounding factors (Valdes et al. 2010a). Genetic factors influence not only OA onset, but also disease severity or progression and outcomes of OA at various stages during the course of the disease. Classic twin studies and familial aggregation studies have also investigated the genetic contribution to longitudinal changes in knee structure, cartilage volume and radiographic progression of OA and showed that all these traits have a substantial heritability, ranging from 33% for change in lateral knee osteophyte grade to 73% for change in medial cartilage volume (Valdes et al. 2010a).

Over the last years, the development of high throughput microarray-based single nucleotide polymorphisms (SNPs) genotyping techniques and the genome-wide association studies (GWAS) have helped to discover genetic markers, mainly SNPs, associated with knee OA susceptibility and OA progression or severity. SNPs in genes, such as GDF5, EDG2 or DVWA, among others, have been described as associated to knee OA susceptibility (Valdes et al. 2011; Mototani et al. 2008; Evangelou et al. 2011). An SNP-based haplotype in the IL-1RA gene and a SNP in the ADAM12 gene have been found to be associated to radiographic severity or progression of knee OA (Attur et al., 2010; Kerkhof et al. 2011; Kerna et al. 2009), and a SNP in the TP63 gene has been suggested as probably associated to total knee replacement (ARCOGEN study 2012).

The clinical course of knee OA is highly variable. Some patients remain without significant functional loss and/or radiological damage progression for many years, while others become impaired or need an arthroplasty (knee replacement) within a few years since disease onset. Predicting the course of knee OA in each patient could aid the clinician in the management of the disease, allowing for personalized medicine based on choosing the most suitable therapeutic strategy for each patient from early stages of the disease.

It has been suggested that combinations of genetic markers, or genetic markers with clinical or demographic variables, could be used to identify individuals at high risk of OA, risk of total joint arthroplasty failure or risk of developing a more severe knee OA phenotype, which should facilitate the application of preventive and disease management strategies (Valdes et al. 2010a, Valdes et al. 2010b; Attur et al., 2010). However, up to date, there are few studies analyzing, or which have found combinations of genetic markers for predicting knee OA severity. Specifically, Kerkhof et al. have filed a patent application for a method for detection of the risk for developing OA or progression of OA comprising detecting the presence of one or more single nucleotide polymorphisms (SNPs) selected from an specific group of several SNPs (Patent application: WO2010071405). Dietrich et al. have filed a patent application for a method for assessing phenotypes, such as OA susceptibility or prognosis, of an individual's genomic information, such as single nucleotide polymorphisms (SNPs), comprises comparing a genomic profile of the individual with a database of genotype/phenotype correlations; combining multiple genetic markers, together with other information, to produce a Genetic Composite Index (GCI) score (Patent application: WO2008067551)

There remains a clear need for methods of predicting severity or progression to knee OA based on genetic markers. The present invention addresses this need among others.

### DISCLOSURE OF THE INVENTION

The present inventors have surprisingly found that combinations of genomic markers as defined herein are able to provide accurate predictions of the severity of osteoarthritis (OA), particularly the progression of OA to a more severe phenotype. Specific risk alleles and risk genotypes at each of the identified positions of single nucleotide polymorphism (SNP) combine to provide accuracy that makes the prediction of, e.g., radiographic progression of knee OA informative, e.g., for treatment and clinical decision making. As described in detail herein, the “gold standard” of accuracy of prediction, being an area under the curve (AUC) of a receiver operating characteristic (ROC) curve of at least 0.7 is demonstrated for a large number of combinations of at least 4 SNPs as set forth in Table 2. Moreover, the set of 23 SNPs associated with radiographic knee OA prognosis (set forth in Table 1) are unified by a common special technical feature; that is to say, this group of SNPs combine in sets of at least 4 to provide a high level of accuracy of prediction (AUC-ROC≧0.7), whereas sets of at least 4 SNPs which include SNPs that are not among those in Table 1 fail to reach an AUC-ROC of ≧0.7 (see, e.g., Table 6). This is the case even when only one SNP in a set of four is replaced with a SNP from outside of Table 1, and even when the replacement SNP is itself associated with radiographic knee OA progression at the genotypic level (see Tables 7 and 11 herein). Without wishing to be bound by any particular theory, the present inventors believe that the SNPs of Table 1 form a “unified web” of markers for OA progression that are unusually effective in their predictive accuracy.

Accordingly, in a first aspect the present invention provides a method for predicting the severity or progression of osteoarthritis (OA) in a human subject, comprising: determining the identity of at least one allele at each of at least 4 (such as at least 5, at least 6, at least 7, at least 8, at least 9 or at least 10) positions of single nucleotide polymorphism (SNPs) selected from the group consisting of: rs2206593, rs10465850, rs780094, rs1374281, rs1143634, rs2073508, rs2243250, rs4720262, rs917760, rs7838918, rs12009, rs730720, rs874692, rs893953, rs1799750, rs10845493, rs11054704, rs7986347, rs1802536, rs10519263, rs7342880, rs16947882 and rs10413815, and one or more SNPs in linkage disequilibrium (LD) at a level of at least R²≧0.8 therewith.

The skilled person is readily able to determine whether a given SNP is in (LD) with a SNP set forth in Table 1 at a level of at least R²≧0.8. Indeed, R²≧0.8 is a well-established threshold of LD described in the literature (see, e.g., Carlson et al., 2004, Am. J. Hum. Genet. 74:106-120). Carlson et al. describe testing different threshold values for R², and established that R²≧0.8 is the best-suited for establishing TagSNPs, as it resolved most of the haplotypes. The scientific basis that underlies linkage disequilibrium makes it clear that a SNP that has R²≧0.8 with a SNP set forth in Table 1 will, like the Table 1 SNP itself, be associated with the prognosis of OA (in particular, knee OA progression) to a significant degree. However, in certain preferred cases, the at least 4 SNPs are selected from the group consisting of: rs2206593, rs10465850, rs780094, rs1374281, rs1143634, rs2073508, rs2243250, rs4720262, rs917760, rs7838918, rs12009, rs730720, rs874692, rs893953, rs1799750, rs10845493, rs11054704, rs7986347, rs1802536, rs10519263, rs7342880, rs16947882 and rs10413815.

As shown in Table 1, at each SNP a particular allele is identified as being a “risk” allele in that it increases the likelihood that the subject carrying said allele will suffer progression of OA to a more severe phenotype. Likewise, at each SNP a particular genotype or pair of genotypes (e.g. homozygous for the risk allele, and in some cases heterozygous) is or are identified as being a “risk” genotypes that increase the likelihood that the subject carrying said genotype or genotypes will suffer progression of OA to a more severe phenotype. Therefore, in certain cases in accordance with the method of this and other aspects of the present invention the presence of 1, 2, 3 or 4 or more of the following risk alleles indicates an increased probability of progression of OA in said subject:
rs2206593 T;
rs10465850 C;
rs780094 C;
rs1374281 G;
rs1143634 G;
rs2073508 C;
rs2243250 G;
rs4720262 A;
rs917760 C;
rs7838918 C;
rs12009 C;
rs730720 G;
rs874692 G;
rs893953 A;
rs1799750 C;
rs10845493 A;
rs11054704 T;
rs7986347 T;
rs1802536 A;
rs10519263 C;
rs7342880 T;
rs16947882 C; and
rs10413815 A.

As the skilled person will appreciate, the absence of risk alleles may itself be informative, in that the subject may be accurately be predicted not to suffer progression of OA to a more severe phenotype.

In certain cases in accordance with the method of this and other aspects of the present invention the method comprises determining the genotype of the subject at each of said at least 4 SNPs, and wherein the presence of 1, 2, 3 or 4 or more of the following genotypes indicates an increased probability of progression of OA in said subject:
rs2206593 TT or TC;
rs10465850 CC;
rs780094 CT or CC;
rs1374281 GG;
rs1143634 GG;
rs2073508 CC;
rs2243250 GG;
rs4720262 AA or GA;
rs917760 CC or CG;
rs7838918 CC;
rs12009 CT or CC;
rs730720 GA or GG;
rs874692 GG;
rs893953 AA or AG;
rs1799750 CC or CT;
rs10845493 AA or AG;
rs11054704 TT or TC;
rs7986347 TT;
rs1802536 AA or AC;
rs10519263 CC or CT;
rs7342880 TT or GT;
rs16947882 CC; and
rs10413815 AA.

In some cases in accordance with the method of this and other aspects of the invention the at least 4 SNPs may comprise at least 5, 6, 7, 8, 9 or at least 10 SNPs. However, the examples herein demonstrate that very accurate prediction may be made without resorting to genotyping excessive numbers of SNPs. Thus, an optimal number of SNPs may be chosen to avoid unnecessary use of time and resources. In particular cases in accordance with the method of this and other aspects of the invention the method comprises determining the identity of the alleles at not more than 15, 14, 13, 12, 11, or not more than 10 SNPs.

In some cases in accordance with the method of this and other aspects of the invention the area under the curve (AUC) of a receiver operating characteristic (ROC) curve for the prediction of OA progression is at least 0.7, at least 0.8 or at least 0.9.

In some cases in accordance with the method of this and other aspects of the invention the method further comprises obtaining or determining at least one clinical variable of the subject. The use of a multivariate model that combines the genomic markers (SNPs) with clinical risk factors for OA is able to provide highly informative predictions of OA prognosis. In some cases, the at least one clinical variable is selected from the group consisting of: gender, age, age at diagnosis of knee OA, body mass index, presence of other affected joints by OA, and presence of contralateral joint OA. In preferred cases, the clinical variable is the age of the subject in years at the time of diagnosis of OA, e.g., knee OA. The clinical variable age at diagnosis of OA, e.g. knee OA, may be represented as a binary outcome, wherein age of ≦60 years is one outcome (e.g. value 0) and age >60 is the other outcome (e.g. value 1) (see, in particular, Table 14). However, it is specifically contemplated herein that the method of this and other aspects of the invention may comprise making the prediction of OA severity or progression without including any clinical variables in addition to the SNP alleles (see, e.g., the model set forth in Table 12, which achieves very high accuracy using only SNPs).

In some cases in accordance with the method of this and other aspects of the invention the at least 4 SNPs comprise SNPs (i) to (viii):
(i) rs2073508;
(ii) rs10845493;
(iii) rs2206593;
(iv) rs10519263 and/or rs1802536;
(v) rs7342880;
(vi) rs12009;
(vii) rs874692; and
(viii) rs780094.

The SNPs at (iv), rs10519263 and rs1802536, are both located on chromosome 15 and are in LD. Therefore, one of these two SNPs may be selected interchangeably with the other for inclusion in a predictive model of the present invention. Therefore, one of these two SNPs may be selected interchangeably with the other for inclusion in a predictive model of the present invention. In some cases in accordance with this and other aspects of the present invention, the genotype of the subject at each of said SNPs (i) to (viii) is determined.

As shown in Tables 12 and 14, a set of SNPs that includes rs2073508; rs10845493; rs2206593; rs10519263; rs7342880; rs12009; rs874692; and rs780094 provides a predictive model of OA progression, particularly knee OA progression, that exhibits particularly superior accuracy (AUC-ROC in the region of 0.8). Accordingly, the method of the present invention may comprise use of a predictive model as set forth in Table 12 or Table 14 to predict OA, e.g. knee OA, progression in a human subject.

In some cases in accordance with the method of this and other aspects of the invention the prediction of the progression of OA comprises predicting the progression of knee OA. In particular, the method may be for predicting the progression of knee OA to a severity requiring arthroplasty. In some cases the method may be for predicting the progression of knee OA to Kellgren-Lawrence grade 4, e.g. progression from a lower grade (e.g. 2 or 3) to grade 4.

The subject may have been diagnosed as having OA, in particular knee OA or diagnosed or advised that he or she is predisposed to developing OA, in particular knee OA. In some cases the subject may have previously been diagnosed as having knee OA to Kellgren-Lawrence grade 2 or 3.

In some cases in accordance with the method of this and other aspects of the invention the subject is at least 40 or at least 50 years of age.

In some cases in accordance with the method of this and other aspects of the invention the method is for predicting OA progression within 8 years, in particular, predicting that the subject will or is likely to suffer progression of knee OA to a level requiring arthroplasty within 8 years of knee OA diagnosis. However, it is specifically contemplated herein that the method of the invention finds use in providing a positive prognosis, for example that the subject is predicted not to suffer progression of knee OA to a level requiring arthroplasty for a period of at least 8 years from diagnosis of knee OA.

In some cases in accordance with the method of this and other aspects of the invention the method comprises use of a probability function. The probability function may, for example, combine the SNP allele/genotype variables and, where applicable, clinical variables with appropriate weighting given to each variable. In some cases the probability function comprises beta coefficient values as set forth in Table 12 or Table 14 (see the column headed “β” in each of Tables 12 and 14.

The skilled person is readily able to select a suitable technique for determining the identity of the allele(s) at each of said positions of SNP of the subject. Methods for genotyping using for example alleles-specific PCR, allele-specific probe hybridisation, restriction fragment length polymorphism and/or DNA sequencing are well-known in the art and can be adapted readily to interrogating the SNPs set forth in Table 1 for one or a plurality of subjects. Moreover, the method for determining the identity of the allele(s) at each of said positions of SNP of the subject may advantageously comprise a multiplex method wherein two or more SNPs are analysed in parallel. This provides efficiency savings, not least in time and sample processing.

In some cases, determining the identity of said at least one allele at each of said positions of SNP of said subject comprises amplification, hybridization, allele-specific PCR, array analysis, bead analysis, primer extension, restriction analysis and/or sequencing.

In accordance with the method of this and other aspects of the present invention, the method is preferably an in vitro method that is carried out on a sample (e.g. a biological liquid, cell or tissue sample) that has been obtained and/or isolated from the subject. However, in some cases it is specifically contemplated that the method may additionally comprise a preceding step of obtaining a sample, in particular a DNA-containing sample, from the subject.

In some cases in accordance with the method of this and other aspects of the invention the sample is selected from the group consisting of: blood, skin cells, cheek cells, saliva, hair follicles, and tissue biopsy.

In some preferred cases in accordance with the method of this and other aspects of the present invention, determining the identity of the at least one allele at each of said at least 4 positions of SNP of said subject comprises:
extracting genomic DNA from a sample obtained from the subject;
amplifying portions of genomic DNA by PCR, wherein the portions of genomic DNA comprise said at least 4 SNPs, and wherein the PCR products are biotinylated during the PCR process;
hybridizing the PCR products to DNA probes which probes are conjugated to microbeads;
fluorescently labelling the hybridized DNA;
analysing the fluorescence signals of the labelled DNA using a microbead fluorescence reader to determine the identity of one or both alleles at each of said positions of SNP; and
predicting the likelihood of OA progression based on the identity of one or both alleles at each of said positions of SNP. In certain cases, a programmable computer is used to predict the likelihood of OA progression based on the identity of one or both alleles at each of said positions of SNP. Specifically contemplated herein is a method wherein computer software is used to automate or semi-automate the process of deriving a prediction of OA progression from the SNP allele identity results, thereby minimising individual operator bias. Moreover, the use of a computer-assisted method of analysis provides speed and efficiency of operation.

In a second aspect, the present invention provides a method for treating osteoarthritis (OA), in particular knee OA, in a human subject, comprising:
(i) carrying out the method of the first aspect of the invention on a sample obtained from the subject; and
(ii) using the prediction of OA progression, particularly knee OA, determined in (i) to select a treatment regimen for therapy of OA, particularly knee OA, of the subject,
wherein a treatment regimen is selected when progression of OA, particularly knee OA, is predicted. Treatment of OA may include one or more of: physical therapy, use of orthoses, non-steroidal anti-inflammatory drugs (NSAIDs), COX-2 selective inhibitors, analgesics, opioid analgesics, glucocorticoids, glycosaminoglycans, amino sugars and surgery.

In a third aspect, the present invention provides a method for selecting a treatment for osteoarthritis (OA), in particular knee OA, in a human subject, comprising:
(i) carrying out the method of the first aspect of the invention on a sample obtained from the subject; and
(ii) using the prediction of OA progression, particularly knee OA, determined in (i) to select a treatment regimen for therapy of OA, particularly knee OA, of the subject,
wherein a treatment regimen is selected when progression of OA, particularly knee OA, is predicted. Treatment of OA may include one or more of: physical therapy, use of orthoses, non-steroidal anti-inflammatory drugs (NSAIDs), COX-2 selective inhibitors, analgesics, opioid analgesics, glucocorticoids, glycosaminoglycans, amino sugars and surgery.

In a fourth aspect, the present invention provides a method of stratifying a plurality of human subjects according their likelihood of osteoarthritis (OA) progression, the method comprising carrying out the method of the first aspect of the invention on a plurality of subjects and using the prediction of OA progression for each of said plurality to stratify the plurality into at least two strata of OA progression prognosis.

In a fifth aspect, the present invention provides a system for predicting the severity or progression of osteoarthritis (OA) in a human subject, comprising:
a plurality of oligonucleotide probes that interrogate at least 4 positions of single nucleotide polymorphism (SNP) as set forth in Table 1;
at least one detector arranged to detect a signal from detectably labelled DNA obtained from the subject or a detectably labelled amplicon amplified from DNA obtained from the subject;
at least one controller in communication with the at least one detector, the controller being programmed with computer-readable instructions to transform said signal into predicted allele identifications at said positions of SNP, and optionally, to transform said predicted allele identifications into a predicted likelihood of OA progression. In some cases, the detector comprises a microbead fluorescence reader.

The invention will now be described in more detail, by way of example and not limitation, by reference to the accompanying drawings. Many equivalent modifications and variations will be apparent to those skilled in the art when given this disclosure. Accordingly, the exemplary embodiments of the invention set forth are considered to be illustrative and not limiting. Various changes to the described embodiments may be made without departing from the scope of the invention. All documents cited herein are expressly incorporated by reference.

### DESCRIPTION OF THE FIGURES

FIG. 1 shows the AUC-ROC of the predictive models for radiographic KOA prognosis shown in Tables 2, 4, 9 and 10.

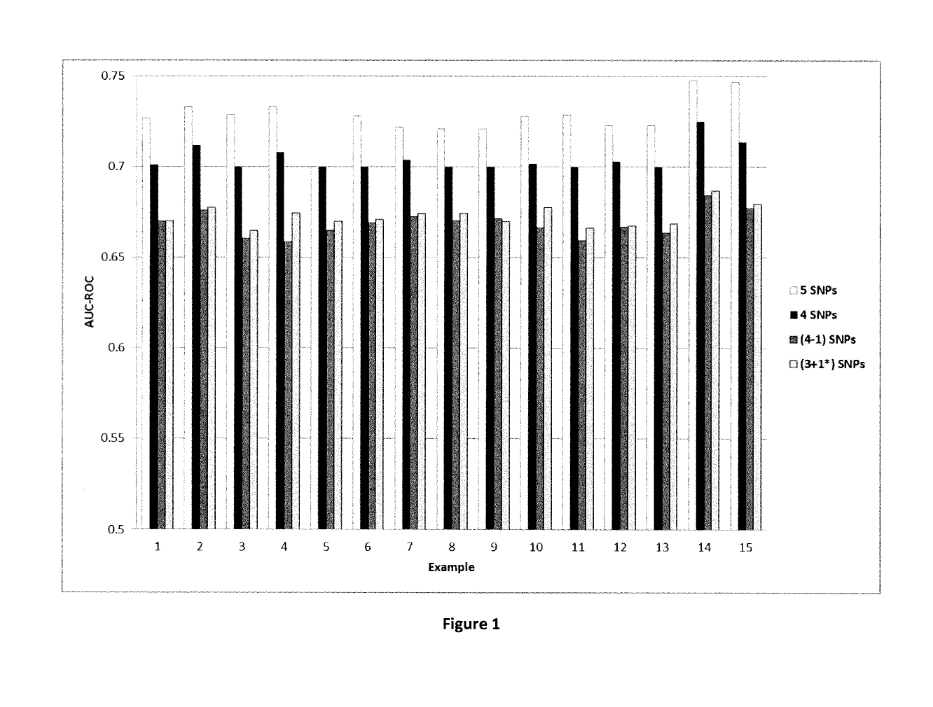

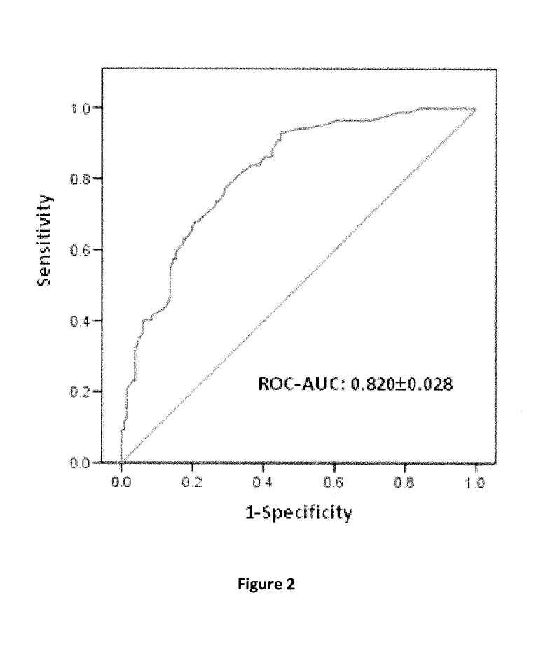

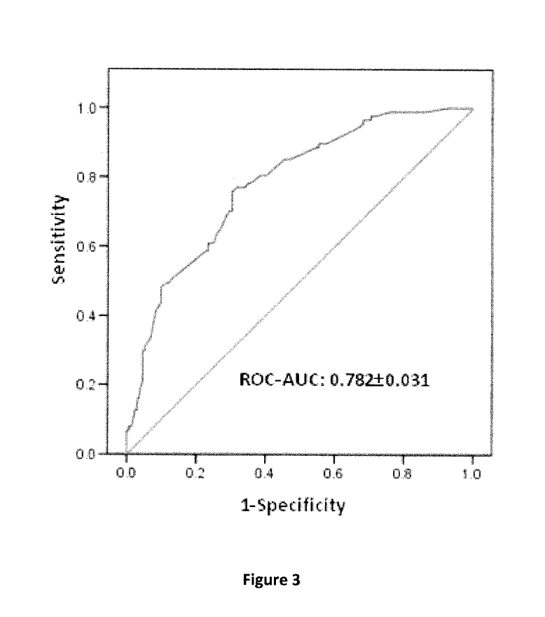

Table 2 includes fifteen examples of predictive models combining 4 SNPs from the list of the 23 associated SNPs to radiographic KOA prognosis. The AUC-ROCs are represented in the FIG. 1 as data entitled: 4 SNPs.

Table 4 includes fifteen examples of predictive models combining 5 SNPs from the list of the 23 associated SNPs to radiographic KOA prognosis. The AUC-ROCs are represented in the FIG. 1 as data entitled: 5 SNPs.

Table 9 includes sixteen examples of predictive models combining 3 SNPs from the list of the 23 associated SNPs to radiographic KOA prognosis. The AUC-ROCs average of the four possible predictive models for each one of the fifteen examples are represented in the FIG. 1 as data entitled: (4-1) SNPs.

Table 10 includes sixteen examples of predictive models combining 3 SNPs from the list of the 23 associated SNPs to radiographic KOA prognosis and 1 SNP different from the mentioned list (from Table 5 which includes SNPs not associated to radiographic KOA prognosis neither at the allelic level nor at the genotypic level). The AUC-ROCs average of the four possible predictive models for each one of the fifteen examples are represented in the FIG. 1 as data entitled: (3+1) SNPs.

FIG. 2 shows the AUC-ROC of the predictive model for radiographic KOA prognosis shown in the Table 12.

FIG. 3 shows the AUC-ROC of the predictive model for radiographic KOA prognosis shown in the Table 14.

### DETAILED DESCRIPTION

As used herein, positions of single nucleotide polymorphism (SNP) are identified by rs number, said rs number denoting the database entry in the NCBI dbSNP build 137, Homo sapiens genome build 37.3, updated 26 Jun. 2012. The entire contents of each rs number entry identified herein, including flanking sequence, is expressly incorporated herein by reference.

### EXAMPLES

Patients

This study was approved by the clinical research ethical committee of the involved Hospitals. Data were collected on patients at departments of rheumatology, orthopaedics, rehabilitation and primary care at 31 Spanish hospitals and primary care centers.

The study population consisted of 219 Knee Osteoarthritis (KOA) patients fulfilling the following eligibility criteria:

Inclusion Criteria:
Patients who had a clinical and radiological diagnosis of primary KOA
Patients who at KOA diagnosis moment were ≧40 years old
Patients who at KOA diagnosis moment had a radiographic Kellgren-Lawrence grade 2 or 3.
Patients with a follow-up since diagnosis of:

a) 8 years or less if had reached a KL grade of 4 or/and have undergone an arthroplasty (bad prognosis). Minimum follow-up of 2 years. 87 out of 219 recruited KOA patients were classified into the bad prognosis group.

b) 8 years or more if had not reached a KL grade of 4 and neither have undergone and arthroplasty (good prognosis). 132 out of 219 recruited KOA patients were classified into the good prognosis group.
Patients with two X-rays: one of the beginning and the other of the end of the follow up period.
Patients who provided saliva or blood sample.
Patients who provided a written informed consent.

Exclusion Criteria:
Patients with KOA secondary to fractures or to metabolic, endocrine or other rheumatic diseases.
Patients not able to understand and cooperate with the requirements of the study protocol.

Besides, an external population was recruited. The external population was composed of 62 KOA patients, 37 out of 62 with bad radiographic KOA prognosis and 25 out 62 with good radiographic KOA prognosis.

The study was done in accordance with the Helsinki Declaration and European Medicines Agency recommendations.

Clinical Evaluation

The two X-rays per recruited KOA patient (at the beginning and at the end of the follow-up) were evaluated by the same evaluator in order to avoid bias in the classification of the X-rays into the Kellgren-Lawrence grades.

SNP Selection, DNA Isolation and SNP Genotyping

We followed a candidate gene strategy. To establish the list of candidate genes, we selected genes implicated in the molecular processes involved in OA (cartilage degradation, inflammation, extracellular matrix metabolism and bone remodeling), in genes known to be associated with OA, and in genes known to be associated to OA comorbidities (diabetes type 2, metabolic syndrome, hypercholesterolemia) with the available information up to Jun. 21, 2011. We selected 2 or 3 SNPs per gene and if there was no SNP described inside the gene we selected SNPs in the flanking regions. We used dbSNP (http://www.ncbi.nlm.nih.gov/projects/SNP) database for SNPs selection.

DNA was extracted from blood or saliva using the QIAamp DNA Blood Mini Kit from (Qiagen, Hilden, Del.) and quantified with a NanoDrop ND-1000 spectrophotometer (NanoDrop Technologies, Wilmington, Del.). 768 SNPs were genotyped using a IIlumina Golden Gate Assay (Illumina Inc., San Diego, Calif.) (Fan et al. in Cold Spring Harb Symp Quant Biol. 68:69-78 (2003)), and 6 SNPs were genotyped using the KASPar chemistry (KBioscience, Hertfordshire, UK).

Statistical Analysis

Statistical analyses were performed by using the SPSS v15.0 (SPSS, Chicago, Ill., USA), the HelixTree (Golden Helix, Bozeman, Mont., USA) and the Analyse-it (Analyse-it Software, Ltd., UK) softwares.

Univariate analysis using the chi-square and Student unpaired t tests was done to identify associations between baseline clinical variables (CVs) or genetic polymorphisms (SNPs) and radiographic KOA prognosis. Only SNPs conforming to Hardy-Weinberg expectations in each group were included. For each SNP all inheritance models were explored. To limit the overall false-positive rate variables (SNPs and CVs) were filtered before modeling (Steyerberg E).

Individual association p values were used to rank SNPs, and only SNPs with an association of p<0.05 (Chi-Squared Single Value Permutation test, n=1000 permutations) in the allelic association test and genotypic association test were included on multivariate analysis. Individual association p values were also used to rank CVs (gender, age at KOA diagnosis, body mass index, presence of other affected joints by OA, contralateral joint OA, etc. . . . ), and only CVs with an association of p<0.05 (Chi-Squared test or Student unpaired t test or non-parametric Mann-Whitney test).

Odds ratios (OR) were calculated with 95% confidence intervals (CI).

Multivariate analysis or predictive models were done using forward RV logistic regression. Radiographic KOA progression was considered the dependent variable, and baseline CVs and SNPs were included as predictors. Each SNP was included, considering the inheritance model significantly associated with the phenotype. The p values to enter and remove cutoffs were 0.05 and 0.1, respectively (Steyerberg E).

Accuracy was assessed by the ROC curve AUC. To measure the impact of the SNPs and variables included in the models of the analyzed phenotype, the sensitivity (S), specificity (Sp) and positive likelihood ratio [LR+=sensitivity/(1_specificity)] were computed by means of the ROC curves.

Models were externally validated by using an external population composed of 62 KOA patients (25 out of 62 KOA patients with good radiographic KOA prognosis and 37 out of 62 KOA patients with bad radiographic KOA prognosis). A Z test to compare two independent samples was used to analyse if the observed differences between AUC-ROCs (initial population versus external population) were statistically significant.

Results

SNPs with poor genotype cloud clustering or <90% (18 and 6 SNPs, respectively) and those which were not in Hardy-Weinberg equilibrium in the population (p<0.0001) (3 SNPs) were excluded. Monomorphic SNPs were also excluded (33 SNPs). We also excluded samples with an individual genotyping call-rate <90% (6 samples were excluded). Therefore, a total of 714 SNPs and 281 samples (219 samples of the initial population and 62 samples of the external population) verified the quality control criteria.

We found a total of 23 SNPs significantly associated to radiographic KOA prognosis at the allelic and genotypic level (single value (SV) permutation allele and genotype test (1000 permutations), p<0.05) in the comparison bad prognosis versus good prognosis. The 23 SNPs associated to radiographic KOA prognosis are displayed in the Table 1.

We found that 2 of the 23 associated SNPs to radiographic KOA prognosis were in strong linkage disequilibrium (R²≧0.8), and therefore these SNPs are not independent variables. The linked SNPs are located in the Chromosome 15, rs1802536 and rs10519263.

Statistical results of allele and genotype comparisons of the 23 SNPs are given in Table 1. In the Table 1 it is specified if the risk allele corresponds to the TOP or BOT strand of the DNA following Ilumina's nomenclature for DNA strand identification. The simplest case of determining strand designations occurs when one of the possible variations of the SNP is an adenine (A), and the remaining variation is either a cytosine (C) or guanine (G). In this instance, the sequence for this SNP is designated TOP. Similar to the rules of reverse complementarity, when one of the possible variations of the SNP is a thymine (T), and the remaining variation is either a C or a G, the sequence for this SNP is designated BOT. If the SNP is an [A/T] or a [C/G], then the above rules do not apply.

Illumina employs a ‘sequence walking’ technique to designate Strand for [A/T] and [C/G] SNPs. For this sequence walking method, the actual SNP is considered to be position ‘n’. The sequences immediately before and after the SNP are ‘n−1’ and ‘n+1’, respectively. Similarly, two base pairs before the SNP is ‘n−2’ and two base pairs after the SNP ‘n+2’, etc. Using this method, sequence walking continues until an unambiguous pairing (A/G, A/C, T/C, or T/G.) is present. To designate strand, when the A or T in the first unambiguous pair is on the 5′ side of the SNP, then the sequence is designated TOP. When the A or T in the first unambiguous pair is on the 3′ side of the SNP, then the sequence is designated BOT.

The codification of the SNPs considering the more significant inheritance model is shown in the Table 1. Besides, the OR (95% CI) is included.

TABLE 1
SNPs associated to radiographic KOA prognosis (23 SNPs). SNP code, chromosome position,
gene, gene region, nucleotide change, risk allele considering Illumina's TOP/BOT strand
nomenclature, allele and genotype association tests results, and Odd Ratio (OR) are shown.

| | | | | Nucleotide | | |
|---|---|---|---|---|---|---|
| | | | | change | | |
| SNP code | | | | [Major/Minor | DNA | |
| (rs) | Chr | Gene | Gene region | allele] | Strand | MAF |
| rs2206593 | 1 | PTGS2 | UTR | [C/T] | BOT | 0.04 |
| rs10465850 | 1 | AK3L1/LOC645195 | intergenic | [C/T] | BOT | 0.45 |
| rs780094 | 2 | GCKR | intron | [C/T] | BOT | 0.45 |
| rs1374281 | 2 | IL1RN/PSD4 | intergenic | [C/G] | BOT | 0.47 |
| rs1143634 | 2 | IL1B | coding (nonsyn) | [G/A] | TOP | 0.21 |
| rs2073508 | 5 | TGFBI | intron | [C/T] | BOT | 0.23 |
| rs2243250 | 5 | IL4 | promoter | [G/A] | TOP | 0.11 |
| rs4720262 | 7 | TXNDC3 | UTR | [G/A] | TOP | 0.28 |
| rs917760 | 7 | PRKAR2B | intron | [G/C] | BOT | 0.50 |
| rs7838918 | 8 | SFRP1 | intron | [G/C] | BOT | 0.41 |
| rs12009 | 9 | GRP78/HSPA5 | UTR | [C/T] | BOT | 0.49 |
| rs730720 | 10 | CHST3 | UTR | [G/A] | TOP | 0.48 |
| rs874692 | 10 | CHST3 | intron | [G/A] | TOP | 0.20 |
| rs893953 | 11 | B3GAT1 | UTR | [G/A] | TOP | 0.21 |
| rs1799750 | 11 | LOC100289645/MMP1 | intergenic | [C/T] | BOT | 0.50 |
| rs10845493 | 12 | LRP6 | intron | [G/A] | TOP | 0.12 |
| rs11054704 | 12 | BCL2L14/LRP6 | intergenic | [C/T] | BOT | 0.14 |
| rs7986347 | 13 | POSTN | intron | [C/T] | BOT | 0.45 |
| rs1802536 | 15 | LOC100288779/SLC27A2 | complex | [C/A] | TOP | 0.15 |
| rs10519263 | 15 | SLC27A2/LOC100288779 | intergenic | [T/C] | BOT | 0.15 |
| rs7342880 | 17 | TIMP2 | intron | [G/T] | BOT | 0.06 |
| rs16947882 | 17 | SMURF2 | intron | [C/G] | BOT | 0.08 |
| rs10413815 | 19 | HAS1/LOC100287831 | intergenic | [G/A] | TOP | 0.18 |

| | | | Chi- | Codification | |
|---|---|---|---|---|---|
| | | | Squared SV | considering the | |
| | | Chi-Squared SV | Perm. P, | more significant | |
| SNP code | Risk | Perm. P, | Genotype | inheritance | |
| (rs) | allele | Allele Test | Test | model | OR (95% CI) |
| rs2206593 | T | 0.020 | 0.020 | TT, TC vs CC | 3.64 (1.33-9.98) |
| rs10465850 | C | 0.037 | 0.028 | CC vs CT, TT | 2.16 (1.21-3.87) |
| rs780094 | C | 0.010 | 0.027 | CT, CC vs TT | 2.33 (1.11-4.91) |
| rs1374281 | G | 0.014 | 0.028 | GG vs GC, CC | 2.23 (1.14-4.38) |
| rs1143634 | G | 0.024 | 0.032 | GG vs GA, AA | 2.12 (1.19-3.80) |
| rs2073508 | C | 0.002 | 0.004 | CC vs TT, TC | 2.82 (1.56-5.10) |
| rs2243250 | G | 0.030 | 0.030 | GG vs GA, AA | 2.17 (1.07-4.40) |
| rs4720262 | A | 0.018 | 0.047 | AA, GA vs GG | 1.63 (0.95-2.80) |
| rs917760 | C | 0.019 | 0.032 | CC, CG vs GG | 2.33 (1.21-4.46) |
| rs7838918 | C | 0.017 | 0.04 | CC vs CG, GG | 2.25 (1.12-4.55) |
| rs12009 | C | 0.037 | 0.04 | CT, CC vs TT | 2.42 (1.20-4.86) |
| rs730720 | G | 0.013 | 0.044 | GA, GG vs AA | 2.23 (1.08-4.60) |
| rs874692 | G | 0.015 | 0.041 | GG vs GA, AA | 1.87 (1.05-3.31) |
| rs893953 | A | 0.004 | 0.009 | AA, AG vs GG | 2.03 (1.16-3.57) |
| rs1799750 | C | 0.030 | 0.033 | CC, CT vs TT | 2.47 (1.25-4.86) |
| rs10845493 | A | 0.002 | 0.006 | AA, AG vs GG | 2.60 (1.36-4.95) |
| rs11054704 | T | 0.019 | 0.015 | TT, TC vs CC | 2.01 (1.09-3.71) |
| rs7986347 | T | 0.031 | 0.003 | TT vs TC, CC | 3.34 (1.62-6.89) |
| rs1802536 | A | 0.013 | 0.021 | AA, AC vs CC | 2.29 (1.25-4.19) |
| rs10519263 | C | 0.013 | 0.022 | CC, CT vs TT | 2.24 (1.23-4.05) |
| rs7342880 | T | 0.019 | 0.019 | TT, GT vs GG | 2.54 (1.08-5.96) |
| rs16947882 | C | 0.044 | 0.044 | CC vs CG, GG | 2.45 (1.05-5.70) |
| rs10413815 | A | 0.045 | 0.026 | AA vs AG, GG | 11.32 (1.37-93.71) |

Multivariate analysis or predictive models were done using forward RV logistic regression. Radiographic KOA progression was considered the dependent variable, and SNPs were included as predictors. Each SNP was included, considering the more significant inheritance model (Table 1).

The accuracy of the predictive models was evaluated by means of the area under the curve (AUC) of a receiver operating characteristic (ROC) curve. The area under the ROC curve (AUC-ROC) is a measure of discrimination; a model with a high area under the ROC curve suggests that the model is able to accurately predict the value of an observation's response (the radiographic KOA progression in our example).

Hosmer and Lemeshow provide general rules for interpreting AUC values. Paraphrasing their rules gives the general guidelines below (Hosmer D W, and Lemeshow S):

AUC=0.5: No discrimination (i.e., might as well flip a coin)

0.7≦AUC<0.8: Acceptable discrimination

0.8≧AUC<0.9: Excellent discrimination

AUC≧0.9: Outstanding discrimination (but extremely rare)

Therefore, we found predictive models or combinations of SNPs with at least an acceptable discrimination for their use in the radiographic, therefore at least with an AUC-ROC≧0.70(≧70%).

Combinations of at least 4 SNPs from the list of the 23 associated SNPs to radiographic KOA prognosis allow to reach AUC-ROCs≧0.70 (70%). We present herein, as non-limiting examples, 15 examples (Table 2). The 23 associated SNPs to radiographic KOA prognosis are represented the number of times indicated in the Table 3. Therefore, each one of the 23 SNPs are included at least once in the 15 examples of predictive models shown in the Table 2.

TABLE 2
Fifteen examples of predictive models combining
4 SNPs from the list of the 23 associated SNPs
(Table 1) to radiographic KOA prognosis.

| | SNPs included in | |
|---|---|---|
| Example | the predictive model | AUC-ROC |
| 1 | rs2073508 | 0.701 |
| | rs2206593 | |
| | rs2243250 | |
| | rs4720262 | |
| 2 | rs7986347 | 0.712 |
| | rs874692 | |
| | rs893953 | |
| | rs917760 | |
| 3 | rs11054704 | 0.700 |
| | rs1143634 | |
| | rs10519263 | |
| | rs780094 | |
| 4 | rs1802536 | 0.708 |
| | rs730720 | |
| | rs7342880 | |
| | rs7838918 | |
| 5 | rs10413815 | 0.700 |
| | rs10465850 | |
| | rs893953 | |
| | rs10845493 | |
| 6 | rs1374281 | 0.700 |
| | rs16947882 | |
| | rs2073508 | |
| | rs1799750 | |
| 7 | rs7986347 | 0.704 |
| | rs874692 | |
| | rs12009 | |
| | rs917760 | |
| 8 | rs730720 | 0.700 |
| | rs1802536 | |
| | rs11054704 | |
| | rs1143634 | |
| 9 | rs11054704 | 0.700 |
| | rs1143634 | |
| | rs10519263 | |
| | rs730720 | |
| 10 | rs11054704 | 0.702 |
| | rs1143634 | |
| | rs10519263 | |
| | rs874692 | |
| 11 | rs1802536 | 0.700 |
| | rs874692 | |
| | rs7342880 | |
| | rs7838918 | |
| 12 | rs874692 | 0.703 |
| | rs1802536 | |
| | rs11054704 | |
| | rs1143634 | |
| 13 | rs2206593 | 0.700 |
| | rs10519263 | |
| | rs874692 | |
| | rs7342880 | |
| 14 | rs1799750 | 0.725 |
| | rs1802536 | |
| | rs2073508 | |
| | rs2206593 | |
| 15 | rs1802536 | 0.714 |
| | rs2073508 | |
| | rs2206593 | |
| | rs2243250 | |

TABLE 3
Number of times that each one of the 23 SNPs is included in the
fifteen examples of predictive models included in the Table 2.

| 23 SNPs | Number of times |
|---|---|
| rs10413815 | 1 |
| rs10465850 | 1 |
| rs10519263 | 4 |
| rs10845493 | 1 |
| rs11054704 | 5 |
| rs1143634 | 5 |
| rs12009 | 1 |
| rs1374281 | 1 |
| rs16947882 | 1 |
| rs1799750 | 2 |
| rs1802536 | 6 |
| rs2073508 | 4 |
| rs2206593 | 4 |
| rs2243250 | 2 |
| rs4720262 | 1 |
| rs730720 | 3 |
| rs7342880 | 3 |
| rs780094 | 1 |
| rs7838918 | 2 |
| rs7986347 | 2 |
| rs874692 | 6 |
| rs893953 | 2 |
| rs917760 | 2 |

The results showed in Table 2 demonstrate that at least 4 SNPs from the list of the 23 associated SNPs (Table 1) to radiographic KOA prognosis reach an AUC-ROC≧0.70. The Table 4 includes fifteen non limiting examples of predictive models including more than 4 SNPs, exactly 5 SNPs, to demonstrate that more than 4 SNPs from the list of the 23 associated SNPs to radiographic KOA prognosis also reach an AUC-ROC≧0.70.

TABLE 4
Fifteen examples of predictive models combining
5 SNPs from the list of the 23 associated SNPs
(Table 1) to radiographic KOA prognosis.

| | SNPs included in | |
|---|---|---|
| Example | the predictive model | AUC-ROC |
| 1 | rs2073508 | 0.727 |
| | rs2206593 | |
| | rs2243250 | |
| | rs4720262 | |
| | rs10413815 | |
| 2 | rs7986347 | 0.733 |
| | rs874692 | |
| | rs893953 | |
| | rs917760 | |
| | rs10465850 | |
| 3 | rs11054704 | 0.729 |
| | rs1143634 | |
| | rs10519263 | |
| | rs780094 | |
| | rs7986347 | |
| 4 | rs1802536 | 0.733 |
| | rs730720 | |
| | rs7342880 | |
| | rs7838918 | |
| | rs10845493 | |
| 5 | rs10413815 | 0.700 |
| | rs10465850 | |
| | rs893953 | |
| | rs10845493 | |
| | rs11054704 | |
| 6 | rs1374281 | 0.728 |
| | rs16947882 | |
| | rs2073508 | |
| | rs1799750 | |
| | rs12009 | |
| 7 | rs7986347 | 0.722 |
| | rs874692 | |
| | rs12009 | |
| | rs917760 | |
| | rs1143634 | |
| 8 | rs730720 | 0.721 |
| | rs1802536 | |
| | rs11054704 | |
| | rs1143634 | |
| | rs893953 | |
| 9 | rs11054704 | 0.721 |
| | rs1143634 | |
| | rs10519263 | |
| | rs730720 | |
| | rs1374281 | |
| 10 | rs11054704 | 0.728 |
| | rs1143634 | |
| | rs10519263 | |
| | rs874692 | |
| | rs2073508 | |
| 11 | rs1802536 | 0.729 |
| | rs874692 | |
| | rs7342880 | |
| | rs7838918 | |
| | rs2243250 | |
| 12 | rs874692 | 0.723 |
| | rs1802536 | |
| | rs11054704 | |
| | rs1143634 | |
| | rs4720262 | |
| 13 | rs2206593 | 0.723 |
| | rs10519263 | |
| | rs874692 | |
| | rs7342880 | |
| | rs780094 | |
| 14 | rs1799750 | 0.748 |
| | rs1802536 | |
| | rs2073508 | |
| | rs2206593 | |
| | rs7342880 | |
| 15 | rs1802536 | 0.747 |
| | rs2073508 | |
| | rs2206593 | |
| | rs2243250 | |
| | rs7838918 | |

Combinations of 4 SNPs different from the ones included in the Table 1 do not reach the AUC-ROC≧0.70. The Table 5 includes 24 SNPs different from the 23 associated SNPs to radiographic KOA prognosis which are not associated to radiographic KOA prognosis neither at the allelic level nor at the genotypic level (single value (SV) permutation allele and genotype test (1000 permutations)). The Table 6 includes six non limiting examples of predictive models combining 4 SNPs (included in the Table 5) different from the 23 associated SNPs to radiographic KOA prognosis.

TABLE 5
SNPs not associated to radiographic KOA prognosis (24 SNPs)
neither at the allelic level nor at the genotypic level.

| | | Chi-Squared |
|---|---|---|
| | Chi-Squared | SV Perm. P, |
| SNP code (rs) | SV Perm. P, | Genotype Test |
| rs1004317 | 1.000 | 0.770 |
| rs10830962 | 1.000 | 1.000 |
| rs1138714 | 0.982 | 0.999 |
| rs11666933 | 0.959 | 0.656 |
| rs11763517 | 1.000 | 0.454 |
| rs11965969 | 0.976 | 0.660 |
| rs12451299 | 1.000 | 0.966 |
| rs1256034 | 1.000 | 0.356 |
| rs1800629 | 1.000 | 0.383 |
| rs2162679 | 1.000 | 0.630 |
| rs2228547 | 1.000 | 0.575 |
| rs2808628 | 1.000 | 0.732 |
| rs3729877 | 1.000 | 0.806 |
| rs3753793 | 1.000 | 0.217 |
| rs3778099 | 1.000 | 0.897 |
| rs4554480 | 1.000 | 0.652 |
| rs4791171 | 1.000 | 0.265 |
| rs6744682 | 1.000 | 0.587 |
| rs6902771 | 1.000 | 1.000 |
| rs7757372 | 1.000 | 1.000 |
| rs886827 | 1.000 | 0.981 |
| rs888186 | 1.000 | 1.000 |
| rs969531 | 1.000 | 0.771 |
| rs996999 | 1.000 | 0.809 |

TABLE 6
Six examples of predictive models combining 4 SNPs from
the Table 5 which are different from the list of the 23
associated SNPs (Table 1) to radiographic KOA prognosis.

| | SNPs included in | |
|---|---|---|
| Example | the predictive model | AUC-ROC |
| 1 | rs886827 | 0.534 |
| | rs2808628 | |
| | rs4791171 | |
| | rs3753793 | |
| 2 | rs11763517 | 0.510 |
| | rs10830962 | |
| | rs1004317 | |
| | rs2162679 | |
| 3 | rs1138714 | 0.536 |
| | rs4554480 | |
| | rs11965969 | |
| | rs11666933 | |
| 4 | rs12451299 | 0.519 |
| | rs1800629 | |
| | rs996999 | |
| | rs6744682 | |
| 5 | rs888186 | 0.522 |
| | rs2228547 | |
| | rs6902771 | |
| | rs3729877 | |
| 6 | rs7757372 | 0.512 |
| | rs3778099 | |
| | rs1256034 | |
| | rs969531 | |

Table 7 includes 10 SNPs different from the 23 associated SNPs to radiographic KOA prognosis which are not associated to radiographic KOA prognosis both at the allelic level and at the genotypic level. These 10 SNPs are only associated to radiographic KOA prognosis at the genotypic level.

TABLE 7
SNPs only associated to radiographic KOA prognosis (10 SNPs)
at the genotypic level (not associated at the allelic level).

| | | Chi-Squared |
|---|---|---|
| | Chi-Squared | SV Perm. P, |
| SNP code (rs) | SV Perm. P, | Genotype Test |
| rs2077119 | 1.000 | 0.010 |
| rs6073718 | 0.918 | 0.014 |
| rs1667290 | 0.849 | 0.008 |
| rs13963 | 0.827 | 0.034 |
| rs2593813 | 0.601 | 0.022 |
| rs3787166 | 0.600 | 0.031 |
| rs4918 | 0.449 | 0.031 |
| rs6930713 | 0.383 | 0.046 |
| rs314751 | 0.354 | 0.044 |
| rs1612691 | 0.309 | 0.009 |

These examples (Table 6 and Table 8) prove that combinations of 4 SNPs different from the list of the 23 associated SNPs (Table 1) do not reach the AUC-ROC≧0.70, neither combinations of 4 SNPs not associated to radiographic KOA prognosis (neither at the allelic level nor at the genotypic level, Table 5) nor combinations of 4 SNPs associated only at the genotypic level (Table 7).

TABLE 8
Three examples of predictive models combining 4 SNPs from
the Table 7 which are different from the list of the 23 associated
SNPs (Table 1) to radiographic KOA prognosis.

| | SNPs included in | |
|---|---|---|
| Example | the predictive model | AUC-ROC |
| 1 | rs2593813 | 0.670 |
| | rs3787166 | |
| | rs4918 | |
| | rs6930713 | |
| 2 | rs2077119 | 0.691 |
| | rs6073718 | |
| | rs1667290 | |
| | rs1612691 | |
| 3 | rs13963 | 0.687 |
| | rs314751 | |
| | rs2593813 | |
| | rs4918 | |

Predictive models including less than 4 SNPs from the list of the 23 associated SNPs (Table 1) to radiographic KOA prognosis do not reach the AUC-ROC≧0.70. The Table 9 includes the four possible predictive models combining 3 SNPs pear each one of the fifteen non limiting examples shown in Table 2.

Predictive models including 4 SNPs, 3 SNPs from the list of the 23 associated SNPs (Table 1) to radiographic KOA prognosis and 1 SNP different from the list of the 23 associated SNPs, do not reach the AUC-ROC≧0.70. The Table 10 includes the four possible predictive models combining 3 SNPs from the list of the 23 associated SNPs and 1 SNP from the Table 5 which includes SNPs different from the mentioned list (SNPs not associated to radiographic KOA prognosis neither at allelic level nor at genotypic level) per each one of the fifteen non limiting examples shown in Table 2. The Table 11 includes three examples combining 3 SNPs from the list of the 23 associated SNPs and 1 SNP from the Table 7 which includes SNPs different from the mentioned list (SNPs associated to radiographic KOA prognosis at the genotypic level, and not associated at the allelic level).

TABLE 9
Sixteen examples of predictive models combining 3 SNPs from the list of the
23 associated SNPs (Table 1) to radiographic KOA prognosis. This Table includes
the four possible predictive models combining 3 SNPs pear each one of the
fifteen examples shown in Table 2.

| | SNPs | | SNPs | | SNPs | | SNPs | | |
|---|---|---|---|---|---|---|---|---|---|
| | included | | included | | included | | included | | Mean |
| | in the | | in the | | in the | | in the | | of the |
| | predictive | AUC- | predictive | AUC- | predictive | AUC- | predictive | AUC- | AUC- |
| Example | model | ROC | model | ROC | model | ROC | model | ROC | ROCs |
| 1 | rs2073508 | 0.686 | rs2073508 | 0.685 | rs2073508 | 0.673 | rs2206593 | 0.635 | 0.670 |
| | rs2206593 | | rs2206593 | | rs2243250 | | rs2243250 | | |
| | rs2243250 | | rs4720262 | | rs4720262 | | rs4720262 | | |
| 2 | rs7986347 | 0.679 | rs7986347 | 0.678 | rs7986347 | 0.693 | rs874692 | 0.654 | 0.676 |
| | rs874692 | | rs874692 | | rs893953 | | rs893953 | | |
| | rs893953 | | rs917760 | | rs917760 | | rs917760 | | |
| 3 | rs11054704 | 0.675 | rs11054704 | 0.648 | rs11054704 | 0.652 | rs1143634 | 0.667 | 0.661 |
| | rs1143634 | | rs1143634 | | rs10519263 | | rs10519263 | | |
| | rs10519263 | | rs780094 | | rs780094 | | rs780094 | | |
| 4 | rs1802536 | 0.654 | rs1802536 | 0.679 | rs1802536 | 0.660 | rs730720 | 0.641 | 0.659 |
| | rs730720 | | rs730720 | | rs7342880 | | rs7342880 | | |
| | rs7342880 | | rs7838918 | | rs7838918 | | rs7838918 | | |
| 5 | rs10413815 | 0.659 | rs10413815 | 0.673 | rs10413815 | 0.651 | rs10465850 | 0.677 | 0.665 |
| | rs10465850 | | rs10465850 | | rs893953 | | rs893953 | | |
| | rs893953 | | rs10845493 | | rs10845493 | | rs10845493 | | |
| 6 | rs1374281 | 0.666 | rs1374281 | 0.648 | rs1374281 | 0.692 | rs16947882 | 0.671 | 0.669 |
| | rs16947882 | | rs16947882 | | rs2073508 | | rs2073508 | | |
| | rs2073508 | | rs1799750 | | rs1799750 | | rs1799750 | | |
| 7 | rs7986347 | 0.678 | rs7986347 | 0.678 | rs7986347 | 0.682 | rs874692 | 0.652 | 0.673 |
| | rs874692 | | rs874692 | | rs12009 | | rs12009 | | |
| | rs12009 | | rs917760 | | rs917760 | | rs917760 | | |
| 8 | rs730720 | 0.657 | rs730720 | 0.681 | rs730720 | 0.670 | rs1802536 | 0.674 | 0.671 |
| | rs1802536 | | rs1802536 | | rs11054704 | | rs11054704 | | |
| | rs11054704 | | rs1143634 | | rs1143634 | | rs1143634 | | |
| 9 | rs11054704 | 0.675 | rs11054704 | 0.670 | rs11054704 | 0.662 | rs1143634 | 0.679 | 0.672 |
| | rs1143634 | | rs1143634 | | rs10519263 | | rs10519263 | | |
| | rs10519263 | | rs730720 | | rs730720 | | rs730720 | | |
| 10 | rs11054704 | 0.675 | rs11054704 | 0.667 | rs11054704 | 0.661 | rs1143634 | 0.663 | 0.667 |
| | rs1143634 | | rs1143634 | | rs10519263 | | rs10519263 | | |
| | rs10519263 | | rs874692 | | rs874692 | | rs874692 | | |
| 11 | rs1802536 | 0.655 | rs1802536 | 0.669 | rs1802536 | 0.660 | rs874692 | 0.654 | 0.660 |
| | rs874692 | | rs874692 | | rs7342880 | | rs7342880 | | |
| | rs7342880 | | rs7838918 | | rs7838918 | | rs7838918 | | |
| 12 | rs874692 | 0.661 | rs874692 | 0.666 | rs874692 | 0.667 | rs1802536 | 0.674 | 0.667 |
| | rs1802536 | | rs1802536 | | rs11054704 | | rs11054704 | | |
| | rs11054704 | | rs1143634 | | rs1143634 | | rs1143634 | | |
| 13 | rs2206593 | 0.663 | rs2206593 | 0.666 | rs2206593 | 0.671 | rs10519263 | 0.655 | 0.664 |
| | rs10519263 | | rs10519263 | | rs874692 | | rs874692 | | |
| | rs874692 | | rs7342880 | | rs7342880 | | rs7342880 | | |
| 14 | rs1799750 | 0.689 | rs1799750 | 0.668 | rs1799750 | 0.690 | rs1802536 | 0.690 | 0.684 |
| | rs1802536 | | rs1802536 | | rs2073508 | | rs2073508 | | |
| | rs2073508 | | rs2206593 | | rs2206593 | | rs2206593 | | |
| 15 | rs1802536 | 0.690 | rs1802536 | 0.678 | rs1802536 | 0.655 | rs2073508 | 0.686 | 0.677 |
| | rs2073508 | | rs2073508 | | rs2206593 | | rs2206593 | | |
| | rs2206593 | | rs2243250 | | rs2243250 | | rs2243250 | | |

TABLE 10
Sixteen examples of predictive models combining 3 SNPs from the list of
the 23 associated SNPs (Table 1) to radiographic KOA prognosis and 1 SNP
different from the mentioned list (marked by an asterisk; from the Table 5
which includes SNPs not associated to radiographic KOA prognosis neither
at the allelic level nor at the genotypic level). This Table includes the
four possible predictive models combining 3 SNPs from the list and 1 SNP
out the list pear each one of the fifteen examples shown in Table 2.

| | SNPs | | SNPs | | SNPs | | SNPs | | Mean |
|---|---|---|---|---|---|---|---|---|---|
| | included | | included | | included | | included | | of the |
| | in the | AUC- | in the | AUC- | in the | AUC- | in the | AUC- | AUC- |
| Example | model | ROC | model | ROC | model | ROC | model | ROC | ROCs |
| 1 | rs2073508 | 0.687 | rs2073508 | 0.682 | rs2073508 | 0.676 | rs2206593 | 0.636 | 0.670 |
| | rs2206593 | | rs2206593 | | rs2243250 | | rs2243250 | | |
| | rs2243250 | | rs4720262 | | rs4720262 | | rs4720262 | | |
| | rs886827* | | rs886827* | | rs886827* | | rs886827* | | |
| 2 | rs7986347 | 0.680 | rs7986347 | 0.677 | rs7986347 | 0.692 | rs874692 | 0.661 | 0.678 |
| | rs874692 | | rs874692 | | rs893953 | | rs893953 | | |
| | rs893953 | | rs917760 | | rs917760 | | rs917760 | | |
| | rs2808628* | | rs2808628* | | rs2808628* | | rs2808628* | | |
| 3 | rs11054704 | 0.682 | rs11054704 | 0.655 | rs11054704 | 0.651 | rs1143634 | 0.671 | 0.665 |
| | rs1143634 | | rs1143634 | | rs10519263 | | rs10519263 | | |
| | rs10519263 | | rs780094 | | rs780094 | | rs780094 | | |
| | rs4791171* | | rs4791171* | | rs4791171* | | rs4791171* | | |
| 4 | rs1802536 | 0.671 | rs1802536 | 0.690 | rs1802536 | 0.682 | rs730720 | 0.655 | 0.675 |
| | rs730720 | | rs730720 | | rs7342880 | | rs7342880 | | |
| | rs7342880 | | rs7838918 | | rs7838918 | | rs7838918 | | |
| | rs3753793* | | rs3753793* | | rs3753793* | | rs3753793* | | |
| 5 | rs10413815 | 0.664 | rs10413815 | 0.681 | rs10413815 | 0.656 | rs10465850 | 0.677 | 0.670 |
| | rs10465850 | | rs10465850 | | rs893953 | | rs893953 | | |
| | rs893953 | | rs10845493 | | rs10845493 | | rs10845493 | | |
| | rs11763517* | | rs11763517* | | rs11763517* | | rs11763517* | | |
| 6 | rs1374281 | 0.668 | rs1374281 | 0.643 | rs1374281 | 0.693 | rs16947882 | 0.681 | 0.671 |
| | rs16947882 | | rs16947882 | | rs2073508 | | rs2073508 | | |
| | rs2073508 | | rs1799750 | | rs1799750 | | rs1799750 | | |
| | rs1004317* | | rs1004317* | | rs1004317* | | rs1004317* | | |
| 7 | rs7986347 | 0.680 | rs7986347 | 0.680 | rs7986347 | 0.684 | rs874692 | 0.653 | 0.674 |
| | rs874692 | | rs874692 | | rs12009 | | rs12009 | | |
| | rs12009 | | rs917760 | | rs917760 | | rs917760 | | |
| | rs888186* | | rs888186* | | rs888186* | | rs888186* | | |
| 8 | rs730720 | 0.667 | rs730720 | 0.689 | rs730720 | 0.663 | rs1802536 | 0.679 | 0.675 |
| | rs1802536 | | rs1802536 | | rs11054704 | | rs11054704 | | |
| | rs11054704 | | rs1143634 | | rs1143634 | | rs1143634 | | |
| | rs2162679* | | rs2162679* | | rs2162679* | | rs2162679* | | |
| 9 | rs11054704 | 0.680 | rs11054704 | 0.666 | rs11054704 | 0.654 | rs1143634 | 0.679 | 0.670 |
| | rs1143634 | | rs1143634 | | rs10519263 | | rs10519263 | | |
| | rs10519263 | | rs730720 | | rs730720 | | rs730720 | | |
| | rs1138714* | | rs1138714* | | rs1138714* | | rs1138714* | | |
| 10 | rs11054704 | 0.687 | rs11054704 | 0.679 | rs11054704 | 0.671 | rs1143634 | 0.674 | 0.678 |
| | rs1143634 | | rs1143634 | | rs10519263 | | rs10519263 | | |
| | rs10519263 | | rs874692 | | rs874692 | | rs874692 | | |
| | rs4554480* | | rs4554480* | | rs4554480* | | rs4554480* | | |
| 11 | rs1802536 | 0.672 | rs1802536 | 0.669 | rs1802536 | 0.657 | rs874692 | 0.668 | 0.667 |
| | rs874692 | | rs874692 | | rs7342880 | | rs7342880 | | |
| | rs7342880 | | rs7838918 | | rs7838918 | | rs7838918 | | |
| | rs11965969* | | rs11965969* | | rs11965969* | | rs11965969* | | |
| 12 | rs874692 | 0.660 | rs874692 | 0.663 | rs874692 | 0.668 | rs1802536 | 0.679 | 0.668 |
| | rs1802536 | | rs1802536 | | rs11054704 | | rs11054704 | | |
| | rs11054704 | | rs1143634 | | rs1143634 | | rs1143634 | | |
| | rs11666933* | | rs11666933* | | rs11666933* | | rs11666933* | | |
| 13 | rs2206593 | 0.661 | rs2206593 | 0.665 | rs2206593 | 0.680 | rs10519263 | 0.669 | 0.669 |
| | rs10519263 | | rs10519263 | | rs874692 | | rs874692 | | |
| | rs874692 | | rs7342880 | | rs7342880 | | rs7342880 | | |
| | rs12451299* | | rs12451299* | | r512451299* | | rs12451299* | | |
| 14 | rs1799750 | 0.688 | rs1799750 | 0.677 | rs1799750 | 0.693 | rs1802536 | 0.690 | 0.689 |
| | rs1802536 | | rs1802536 | | rs2073508 | | rs2073508 | | |
| | rs2073508 | | rs2206593 | | rs2206593 | | rs2206593 | | |
| | rs996999* | | rs996999* | | rs996999* | | rs996999* | | |
| 15 | rs1802536 | 0.690 | rs1802536 | 0.678 | rs1802536 | 0.660 | rs2073508 | 0.690 | 0.681 |
| | rs2073508 | | rs2073508 | | rs2206593 | | rs2206593 | | |
| | rs2206593 | | rs2243250 | | rs2243250 | | rs2243250 | | |
| | rs3729877* | | rs3729877* | | rs3729877* | | rs3729877* | | |

TABLE 11
Ten examples of predictive models combining 3 SNPs from the
list of the 23 associated SNPs (Table 1) to radiographic
KOA prognosis and 1 SNP different from the mentioned list
(marked by an asterisk; from the Table 7 which includes SNPs
only associated to radiographic KOA prognosis at the genotypic
level, and not associated at the allelic level).

| | SNPs included | |
|---|---|---|
| Example | in the model | AUC-ROC |
| 1 | rs2206593 | 0.680 |
| | rs2243250 | |
| | rs4720262 | |
| | rs2077119* | |
| 2 | rs874692 | 0.693 |
| | rs893953 | |
| | rs917760 | |
| | rs6073718* | |
| 3 | rs11054704 | 0.679 |
| | rs10519263 | |
| | rs780094 | |
| | rs1667290* | |
| 4 | rs10413815 | 0.693 |
| | rs893953 | |
| | rs10845493 | |
| | rs13963* | |
| 5 | rs1374281 | 0.696 |
| | rs16947882 | |
| | rs1799750 | |
| | rs2593813* | |
| 6 | rs874692 | 0.690 |
| | rs12009 | |
| | rs917760 | |
| | rs3787166* | |
| 7 | rs730720 | 0.678 |
| | rs1802536 | |
| | rs11054704 | |
| | rs4918* | |
| 8 | rs11054704 | 0.682 |
| | rs1143634 | |
| | rs730720 | |
| | rs6930713* | |
| 9 | rs11054704 | 0.693 |
| | rs10519263 | |
| | rs874692 | |
| | rs314751* | |
| 10 | rs730720 | 0.687 |
| | rs7342880 | |
| | rs7838918 | |
| | rs1612691* | |

Based on the results showed in Tables 2-11, we can conclude that a combination of at least 4 SNPs from the list of the 23 associated SNPs (Table 1) to radiographic KOA progression allow predictive models with an acceptable AUC-ROC following Hosmer and Lemeshow criteria (Hosmer D W, and Lemeshow 5) (AUC-ROC≧0.70). Both predictive models with 3 SNPs from the 23 associated SNPs and predictive models with 3 SNPs from the 23 associated SNPS plus 1 SNP out of the mentioned SNP list (Table 5 or Table 7) showed AUC-ROCs<0.70, both if the 1 additional SNP out of the list is not associated to radiographic KOA progression neither at the allelic level nor genotypic level and if the 1 additional SNP is only associated at the genotypic level and not at the allelic level. Predictive models with more than 4 SNPs (5 SNPs) from the list of the 23 associated SNPs (Table 1) also showed AUC-ROC≧0.70. These results are summarized in the FIG. 1.

Multivariate analysis or predictive models were done using forward RV logistic regression. Radiographic KOA progression was considered the dependent variable, and SNPs were included as predictors. Each SNP was included, considering the inheritance model significantly associated with the phenotype. The 23 associated SNPs (Table 1) to radiographic KOA progression were included as independent variables. We present herein, as non-limiting examples, a predictive model with an excellent accuracy for radiographic KOA progression which combines 8 SNPs (AUC-ROC over 80%, excellent discrimination following the Hosmer and Lemeshow's general rules for interpreting AUC-ROC values (Hosmer D W, and Lemeshow S) (Table 12 and FIG. 2). The predictive model shows an AUC-ROC of 0.782±0.031 (AUC-ROC±Std. Error), with cut-off points which maximise the sensitivity and specificity of 75.9% and 69.7% respectively. The sensitivity and specificity values at different cut-off points of positive Likelihood Ratio (LR+) are shown in Table 13.

TABLE 12
Example of predictive model for radiographic KOA
progression which combines 8 SNPs.

| | Genotype | | | | | | |
|---|---|---|---|---|---|---|---|
| Variables | or | | | | | | |
| included in | Phenotype | | Std. | | | | |
| the model | Risk | β | Error | P-value | OR | OR, 95% CI | |
| rs2073508 | CC | 1.175 | 0.347 | 0.001 | 3.237 | 1.641 | 6.385 |
| rs10845493 | AA, AG | 0.980 | 0.381 | 0.010 | 2.665 | 1.263 | 5.623 |
| rs2206593 | TT, TC | 1.828 | 0.613 | 0.003 | 6.223 | 1.870 | 20.707 |
| rs10519263 | CC, TC | 0.835 | 0.357 | 0.019 | 2.306 | 1.146 | 4.640 |
| rs7342880 | TT, GT | 1.314 | 0.507 | 0.010 | 3.722 | 1.377 | 10.059 |
| rs12009 | CC, TC | 0.930 | 0.398 | 0.019 | 2.536 | 1.163 | 5.528 |
| rs874692 | GG | 0.952 | 0.347 | 0.006 | 2.590 | 1.312 | 5.111 |
| rs780094 | TT | 0.883 | 0.424 | 0.037 | 2.419 | 1.054 | 5.549 |

TABLE 13
Sensitivity and specificity values at different LR+
cut-off points of the predictive model showed in the Table 12.

| Positive Likelihood Ratio | | |
|---|---|---|
| (LR+) | Sensitivity, % | Specificity, % |
| 2 | 80.5 | 59.9 |
| 5 | 41.3 | 91.7 |
| 10 | 8.1 | 99.2 |

Univariate analysis identified the association between the baseline CV age at KOA diagnosis and the radiographic KOA prognosis. Multivariate analysis or predictive models were done using forward RV logistic regression. Radiographic KOA progression was considered the dependent variable, and SNPs and baseline CVs were included as predictors. Each SNP was included, considering the inheritance model significantly associated with the phenotype. The age at KOA diagnosis was codified as >60 years old versus ≦60 years old. The 23 associated SNPs (Table 1) to radiographic KOA progression were included as independent variables. We present herein, as non-limiting examples, a predictive model with an excellent accuracy for radiographic KOA progression which combines 8 SNPs and 1 CV (AUC-ROC over 80%, excellent discrimination following the Hosmer and Lemeshow's general rules for interpreting AUC-ROC values (Hosmer D W, and Lemeshow S) (Table 14 and FIG. 3). The predictive model shows an AUC-ROC of 0.820±0.028 (AUC-ROC±Std. Error), with cut-off points which maximise the sensitivity and specificity of 73.6% and 73.5% respectively. The sensitivity and specificity values at different cut-off points of positive Likelihood Ratio (LR+) are shown in Table 15.

TABLE 14
Example of predictive model for radiographic
KOA progression which combines 8 SNPs and 1
clinical variable.

| | Genotype | | | | | | |
|---|---|---|---|---|---|---|---|
| Variables | or | | | | | | |
| included in | Phenotype | | Std. | | | | |
| the model | Risk | β | Error | P-value | OR | OR, 95% CI | |
| rs2073508 | CC | 1.160 | 0.360 | 0.001 | 3.190 | 1.576 | 6.457 |
| rs10845493 | AA, AG | 1.021 | 0.407 | 0.012 | 2.775 | 1.250 | 6.160 |
| rs2206593 | TT, TC | 1.491 | 0.594 | 0.012 | 4.440 | 1.386 | 14.225 |
| rs10519263 | CC, TC | 0.732 | 0.374 | 0.050 | 2.080 | 0.999 | 4.329 |
| rs7342880 | TT, GT | 1.081 | 0.523 | 0.039 | 2.947 | 1.057 | 8.220 |
| rs12009 | CC, TC | 1.107 | 0.413 | 0.007 | 3.025 | 1.346 | 6.798 |
| rs874692 | GG | 0.860 | 0.359 | 0.017 | 2.363 | 1.170 | 4.773 |
| rs780094 | TT | 0.930 | 0.446 | 0.037 | 2.535 | 1.058 | 6.071 |
| Age at | >60 years | 1.297 | 0.343 | <0.001 | 3.658 | 1.866 | 7.172 |
| KOA | old | | | | | | |
| diagnosis | | | | | | | |

TABLE 15
Sensitivity and specificity values at different LR+
cut-off points of the predictive model showed in the Table 14.

| Positive Likelihood Ratio | | |
|---|---|---|
| (LR+) | Sensitivity, % | Specificity, % |
| 2 | 93.1 | 54.5 |
| 5 | 41.4 | 91.7 |
| 10 | 20.7 | 98.5 |

Both predictive models (Table 12 and Table 14) were validated in an external KOA population composed of 62 KOA patients, 37 out of 62 with bad radiographic KOA prognosis and 25 out 62 with good radiographic KOA prognosis. Both models were externally validated.

The AUC-ROCs of the model shown in the Table 12 in the initial population (n=219) used to generate the model and in the external population (n=62) were 0.782±0.031 (area±Std. Error) and 0.735±0.067 (area±Std. Error), respectively. There were not statistical differences between these AUC-ROCs (p-value=0.5244). Therefore we can conclude that the predictive model created in an initial population was replicated in an external population.

The AUC-ROCs of the model shown in the Table 14 in the initial population (n=219) used to generate the model and in the external population (n=62) were 0.820±0.028 (area±Std. Error) and 0.726±0.066 (area±Std. Error), respectively. There were not statistical differences between these AUC-ROCs (p-value=0.1898). Therefore we can conclude that the predictive model created in an initial population was replicated in an external population.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, including patent documents, disclosed herein are incorporated by reference in their entirety for all purposes, particularly for the disclosure referenced herein.

### REFERENCES

ARCOGEN (2012). Identification of new susceptibility loci for osteoarthritis (arcOGEN): a genome-wide association study. Lancet, 380, 815-823.

Attur, M., Wang, H. Y., Kraus, V. B., Bukowski, J. F., Aziz, N., Krasnokutsky, S., Samuels, J., Greenberg, J., McDaniel, G., Abramson, S. B. & Kornman, K. S. (2010). Radiographic severity of knee osteoarthritis is conditional on interleukin 1 receptor antagonist gene variations. Ann. Rheum. Dis., 69, 856-861.

Evangelou, E., Valdes, A. M., Kerkhof, H. J., Styrkarsdottir, U., Zhu, Y., Meulenbelt, I., Lories, R. J., Karassa, F. B., Tylzanowski, P., Bos, S. D., Akune, T., Arden, N. K., Carr, A., Chapman, K., Cupples, L. A., Dai, J., Deloukas, P., Doherty, M., Doherty, S., Engstrom, G., Gonzalez, A., Halldorsson, B. V., Hammond, C. L., Hart, D. J., Helgadottir, H., Hofman, A., Ikegawa, S., Ingvarsson, T., Jiang, Q., Jonsson, H., Kaprio, J., Kawaguchi, H., Kisand, K., Kloppenburg, M., Kujala, U. M., Lohmander, L. S., Loughlin, J., Luyten, F. P., Mabuchi, A., McCaskie, A., Nakajima, M., Nilsson, P. M., Nishida, N., Ollier, W. E., Panoutsopoulou, K., van de, P. T., Ralston, S. H., Rivadeneira, F., Saarela, J., Schulte-Merker, S., Shi, D., Slagboom, P. E., Sudo, A., Tamm, A., Tamm, A., Thorleifsson, G., Thorsteinsdottir, U., Tsezou, A., Wallis, G. A., Wilkinson, J. M., Yoshimura, N., Zeggini, E., Zhai, G., Zhang, F., Jonsdottir, I., Uitterlinden, A. G., Felson, D. T., van Meurs, J. B., Stefansson, K., Ioannidis, J. P. & Spector, T. D. (2011). Meta-analysis of genome-wide association studies confirms a susceptibility locus for knee osteoarthritis on chromosome 7q22. Ann. Rheum. Dis., 70, 349-355.

Fernandez-Moreno, M., Rego, I., Carreira-Garcia, V. & Blanco, F. J. (2008). Genetics in osteoarthritis. Curr. Genomics, 9, 542-547.

Kerkhof, H. J., Doherty, M., Arden, N. K., Abramson, S. B., Attur, M., Bos, S. D., Cooper, C., Dennison, E. M., Doherty, S. A., Evangelou, E., Hart, D. J., Hofman, A., Javaid, K., Kerna, I., Kisand, K., Kloppenburg, M., Krasnokutsky, S., Maciewicz, R. A., Meulenbelt, I., Muir, K. R., Rivadeneira, F., Samuels, J., Sezgin, M., Slagboom, E., Smith, A. J., Spector, T. D., Tamm, A., Tamm, A., Uitterlinden, A. G., Wheeler, M., Zhai, G., Zhang, W., van Meurs, J. B. & Valdes, A. M. (2011). Large-scale meta-analysis of interleukin-1 beta and interleukin-1 receptor antagonist polymorphisms on risk of radiographic hip and knee osteoarthritis and severity of knee osteoarthritis. Osteoarthritis. Cartilage., 19, 265-271.

Kerna, I., Kisand, K., Tamm, A. E., Lintrop, M., Veske, K. & Tamm, A. O. (2009). Missense single nucleotide polymorphism of the ADAM12 gene is associated with radiographic knee osteoarthritis in middle-aged Estonian cohort. Osteoarthritis. Cartilage., 17, 1093-1098.

Mototani, H., Iida, A., Nakajima, M., Furuichi, T., Miyamoto, Y., Tsunoda, T., Sudo, A., Kotani, A., Uchida, A., Ozaki, K., Tanaka, Y., Nakamura, Y., Tanaka, T., Notoya, K. & Ikegawa, S. (2008). A functional SNP in EDG2 increases susceptibility to knee osteoarthritis in Japanese. Hum. Mol. Genet., 17, 1790-1797.

Meulenbelt, I., Min, J. L., Bos, S., Riyazi, N., Houwing-Duistermaat, J. J., van der Wijk, H. J., Kroon, H. M., Nakajima, M., Ikegawa, S., Uitterlinden, A. G., van Meurs, J. B., van der Deure, W. M., Visser, T. J., Seymour, A. B., Lakenberg, N., van der, B. R., Kremer, D., van Duijn, C. M., Kloppenburg, M., Loughlin, J. & Slagboom, P. E. (2008). Identification of DIO2 as a new susceptibility locus for symptomatic osteoarthritis. Hum. Mol. Genet., 17, 1867-1875.

Meulenbelt, I. (2012). Osteoarthritis year 2011 in review: genetics. Osteoarthritis. Cartilage., 20, 218-222.

Panoutsopoulou, K., Southam, L., Elliott, K. S., Wrayner, N., Zhai, G., Beazley, C., Thorleifsson, G., Arden, N. K., Carr, A., Chapman, K., Deloukas, P., Doherty, M., McCaskie, A., Ollier, W. E., Ralston, S. H., Spector, T. D., Valdes, A. M., Wallis, G. A., Wilkinson, J. M., Arden, E., Battley, K., Blackburn, H., Blanco, F. J., Bumpstead, S., Cupples, L. A., Day-Williams, A. G., Dixon, K., Doherty, S. A., Esko, T., Evangelou, E., Felson, D., Gomez-Reino, J. J., Gonzalez, A., Gordon, A., Gwilliam, R., Halldorsson, B. V., Hauksson, V. B., Hofman, A., Hunt, S. E., Ioannidis, J. P., Ingvarsson, T., Jonsdottir, I., Jonsson, H., Keen, R., Kerkhof, H. J., Kloppenburg, M. G., Koller, N., Lakenberg, N., Lane, N. E., Lee, A. T., Metspalu, A., Meulenbelt, I., Nevitt, M. C., O'Neill, F., Parimi, N., Potter, S. C., Rego-Perez, I., Riancho, J. A., Sherburn, K., Slagboom, P. E., Stefansson, K., Styrkarsdottir, U., Sumillera, M., Swift, D., Thorsteinsdottir, U., Tsezou, A., Uitterlinden, A. G., van Meurs, J. B., Watkins, B., Wheeler, M., Mitchell, S., Zhu, Y., Zmuda, J. M., Zeggini, E. & Loughlin, J. (2011). Insights into the genetic architecture of osteoarthritis from stage 1 of the arcOGEN study. Ann. Rheum. Dis., 70, 864-867.

Valdes, A. M. & Spector, T. D. (2010a). The clinical relevance of genetic susceptibility to osteoarthritis. Best. Pract. Res. Clin. Rheumatol., 24, 3-14.

Valdes, A. M. & Spector, T. D. (2010b). The genetic epidemiology of osteoarthritis. Curr. Opin. Rheumatol., 22, 139-143.

Valdes, A. M., Evangelou, E., Kerkhof, H. J., Tamm, A., Doherty, S. A., Kisand, K., Tamm, A., Kerna, I., Uitterlinden, A., Hofman, A., Rivadeneira, F., Cooper, C., Dennison, E. M., Zhang, W., Muir, K. R., Ioannidis, J. P., Wheeler, M., Maciewicz, R. A., van Meurs, J. B., Arden, N. K., Spector, T. D. & Doherty, M. (2011). The GDF5 rs143383 polymorphism is associated with osteoarthritis of the knee with genome-wide statistical significance. Ann. Rheum. Dis., 70, 873-875.

Hosmer D W, and Lemeshow S. Applied Logistic Regression. 2nd ed. (2000). John Wiley & Sons, Inc. Pp. 156-164

Steyerberg E. Clinical Prediction Models. A Practical Approach to Development, Validation, and Updating. New York: Springer 2009.

1. A method for predicting the severity or progression of osteoarthritis (OA) in a human subject, comprising: determining the identity of at least one allele at each of at least 4 positions of single nucleotide polymorphism (SNPs) selected from the group consisting of: rs2206593, rs10465850, rs780094, rs1374281, rs1143634, rs2073508, rs2243250, rs4720262, rs917760, rs7838918, rs12009, rs730720, rs874692, rs893953, rs1799750, rs10845493, rs11054704, rs7986347, rs1802536, rs10519263, rs7342880, rs16947882 and rs10413815, and one or more SNPs in linkage disequilibrium at a level of at least R²≧0.8 therewith.

2. A method according to claim 1, wherein said at least 4 SNPs are selected from the group consisting of: rs2206593, rs10465850, rs780094, rs1374281, rs1143634, rs2073508, rs2243250, rs4720262, rs917760, rs7838918, rs12009, rs730720, rs874692, rs893953, rs1799750, rs10845493, rs11054704, rs7986347, rs1802536, rs10519263, rs7342880, rs16947882 and rs10413815.

3. A method according to claim 1 or claim 2, wherein the presence of 1, 2, 3 or 4 or more of the following risk alleles indicates an increased probability of progression of OA in said subject:
rs2206593 T;
rs10465850 C;
rs780094 C;
rs1374281 G;
rs1143634 G;
rs2073508 C;
rs2243250 G;
rs4720262 A;
rs917760 C;
rs7838918 C;
rs12009 C;
rs730720 G;
rs874692 G;
rs893953 A;
rs1799750 C;
rs10845493 A;
rs11054704 T;
rs7986347 T;
rs1802536 A;
rs10519263 C;
rs7342880 T;
rs16947882 C; and
rs10413815 A.

4. A method according to any one of the preceding claims, wherein the method comprises determining the genotype of the subject at each of said at least 4 SNPs, and wherein the presence of 1, 2, 3 or 4 or more of the following genotypes indicates an increased probability of progression of OA in said subject:
rs2206593 TT or TC;
rs10465850 CC;
rs780094 CT or CC;
rs1374281 GG;
rs1143634 GG;
rs2073508 CC;
rs2243250 GG;
rs4720262 AA or GA;
rs917760 CC or CG;
rs7838918 CC;
rs12009 CT or CC;
rs730720 GA or GG;
rs874692 GG;
rs893953 AA or AG;
rs1799750 CC or CT;
rs10845493 AA or AG;
rs11054704 TT or TC;
rs7986347 TT;
rs1802536 AA or AC;
rs10519263 CC or CT;
rs7342880 TT or GT;
rs16947882 CC; and
rs10413815 AA.

5. A method according to any one of the preceding claims, wherein said at least 4 SNPs comprises at least 5, 6, 7, 8, 9 or at least 10 SNPs.

6. A method according to any one of the preceding claims, wherein said method comprises determining the identity of at least one allele at not more than 15, 14, 13, 12, 11, or not more than 10 SNPs.

7. A method according to any one of the preceding claims, wherein the area under the curve (AUC) of a receiver operating characteristic (ROC) curve for the prediction of OA progression is at least 0.7, at least 0.8 or at least 0.9.

8. A method according to any one of the preceding claims, wherein the method further comprises obtaining or determining at least one clinical variable of the subject selected from the group consisting of: gender, age, age at diagnosis of knee OA, body mass index, presence of other affected joints by OA, and presence of contralateral joint OA.

9. A method according to claim 8, wherein said clinical variable comprises the subject's age or age at diagnosis of knee OA.

10. A method according to any one of the preceding claims, wherein said SNPs comprise:
(i) rs2073508;
(ii) rs10845493;
(iii) rs2206593;
(iv) rs10519263 and/or rs1802536;
(v) rs7342880;
(vi) rs12009;
(vii) rs874692; and
(viii) rs780094.

11. A method according to claim 10, wherein the genotype of the subject at each of said SNPs (i) to (viii) is determined.

12. A method according to any one of claims 1 to 11, wherein the prediction of OA progression is based on the model set forth in Table 12.

13. A method according to any one of claims 1 to 11, wherein the prediction of OA progression is based on the model set forth in Table 14.

14. A method according to any one of the preceding claims, wherein said prediction of the progression of OA comprises predicting the progression of knee OA.

15. A method according to claim 14, wherein the method is for predicting the progression of knee OA to a severity requiring arthroplasty.

16. A method according to claim 15, wherein the method is for predicting the progression of knee OA to Kellgren-Lawrence grade 4.

17. A method according to any one of the preceding claims, wherein the subject has been diagnosed as having OA, in particular knee OA.

18. A method according to claim 17, wherein the subject has been diagnosed as having knee OA to Kellgren-Lawrence grade 2 or 3.

19. A method according to any one of the preceding claims, wherein the subject is at least 40 years of age.

20. A method according to any one of the preceding claims, wherein the method is for predicting OA progression within 8 years.

21. A method according to any one of the preceding claims, wherein the subject is predicted to suffer progression of knee OA to a level requiring arthroplasty within 8 years of knee OA diagnosis.

22. A method according to any one of claims 1 to 20, wherein the subject is predicted not to suffer progression of knee OA to a level requiring arthroplasty for a period of at least 8 years from diagnosis of knee OA.

23. A method according to any one of the preceding claims, wherein the subject is predicted to suffer progression of knee OA to Kellgren-Lawrence grade 4 within 8 years of knee OA diagnosis.

24. A method according to any one of claims 1 to 20, wherein the subject is predicted not to suffer progression of knee OA to Kellgren-Lawrence grade 4 for a period of at least 8 years from diagnosis of knee OA.

25. A method according to any one of the preceding claims, wherein the method comprises use of a probability function.

26. A method according to claim 25, wherein the probability function comprises beta coefficient values as set forth in Table 12 or Table 14.

27. A method according to any one of the preceding claims, wherein determining the identity of said at least one allele at each of said positions of SNP of said subject comprises assaying a sample that has previously been obtained from said subject.

28. A method according to claim 27, wherein said sample is selected from the group consisting of: blood, skin cells, cheek cells, saliva, hair follicles, and tissue biopsy.

29. A method according to any one of the preceding claims, wherein determining the identity of said at least one allele at each of said positions of SNP of said subject comprises amplification, hybridization, allele-specific PCR, array analysis, bead analysis, primer extension, restriction analysis and/or sequencing.

30. A method according to any one of the preceding claims, wherein determining the identity of said at least one allele at each of said positions of SNP of said subject comprises:
extracting genomic DNA from a sample obtained from the subject;
amplifying portions of genomic DNA by PCR, wherein the portions of genomic DNA comprise said at least 4 SNPs, and wherein the PCR products are biotinylated during the PCR process;
hybridizing the PCR products to DNA probes which probes are conjugated to microbeads;
fluorescently labelling the hybridized DNA;
analysing the fluorescence signals of the labelled DNA using a microbead fluorescence reader to determine the identity of one or both alleles at each of said positions of SNP; and
predicting the likelihood of OA progression based on the identity of one or both alleles at each of said positions of SNP.

31. A method according to claim 30, wherein a programmable computer is used to predict the likelihood of OA progression based on the identity of one or both alleles at each of said positions of SNP.

32. A method for treating OA in a human subject, comprising:
(i) carrying out a method according to any one of claims 1 to 31 on a sample obtained from the subject;
(ii) using the prediction of OA progression determined in (i) to select a treatment regimen for therapy of OA of the subject,
wherein a treatment regimen is selected when progression of OA is predicted.

33. A method for selecting a treatment for OA in a human subject, comprising:
(i) carrying out a method according to any one of claims 1 to 31 on a sample obtained from the subject;
(ii) using the prediction of OA progression determined in (i) to select a treatment regimen for therapy of OA of the subject,
wherein a treatment regimen is selected when progression of OA is predicted.

34. A method according to claim 32 or claim 33, wherein said OA is knee OA.

35. A method according to any one of claims 32 to 34, wherein said treatment regimen comprises at least one of: physical therapy, use of orthoses, non-steroidal anti-inflammatory drugs (NSAIDs), COX-2 selective inhibitors, analgesics, opioid analgesics, glucocorticoids, glycosaminoglycans, amino sugars and surgery.

36. A method of stratifying a plurality of human subjects according their likelihood of OA progression, the method comprising carrying out a method according to any one of claims 1 to 31 on a plurality of subjects and using the prediction of OA progression for each of said plurality to stratify the plurality into at least two strata of OA progression prognosis.

37. A system for predicting the severity or progression of OA in a human subject, comprising:
a plurality of oligonucleotide probes that interrogate at least 4 positions of single nucleotide polymorphism (SNP) as set forth in Table 1;
at least one detector arranged to detect a signal from detectably labelled DNA obtained from the subject or a detectably labelled amplicon amplified from DNA obtained from the subject;
at least one controller in communication with the at least one detector, the controller being programmed with computer-readable instructions to transform said signal into predicted allele identifications at said positions of SNP, and optionally, to transform said predicted allele identifications into a predicted likelihood of OA progression.

38. A system according to claim 37, wherein said detector comprises a microbead fluorescence reader.
